(19) United States
(12) Patent Application Publication
(10) Pub. No.: US 2015/0094486 A1
(43) Pub. Date: Apr. 2, 2015

(54) ACTIVE IONIC LIQUID MIXTURES FOR OXIDIZING ALKYLAROMATIC COMPOUNDS

(71) Applicant: UOP LLC, Des Plaines, IL (US)

(72) Inventors: Avram M. Buchbinder, Chicago, IL (US); Alakananda Bhattacharyya, Glen Ellyn, IL (US); Praneeth D. Edirisinghe, Chicago, IL (US); Joel T. Walenga, Lake Zurich, IL (US)

(73) Assignee: UOP LLC, Des Plaines, IL (US)

(21) Appl. No.: 14/039,703

(22) Filed: Sep. 27, 2013

(51) Int. Cl. C07C 51/29

(52) U.S. Cl. CPC C07C 51/29; C07C 2101/16
USPC 562/409; 252/364

(57) ABSTRACT

Ionic liquid compositions derived from imidazolium based ionic liquids that have been thermally treated or thermally and oxidatively treated are disclosed. These compositions can be utilized as the medium for oxidation of alkylaromatic compounds and their oxidized derivatives or as a medium for purification of aromatic carboxylic acids.

### BACKGROUND OF THE INVENTION

Oxidation of alkyl aromatic compounds, e.g., toluene and xylenes, are important commercial processes. A variety of oxidation products may be obtained including aromatic carboxylic acids such as terephthalic acid (1,4-benzenedicarboxylic acid) and isophthalic acid (1,3-benzenedicarboxylic acid) which are used, for example, in the polymer industry.

It is known that oxidation products, such as aromatic alcohols, aromatic aldehydes, aromatic ketones, and aromatic carboxylic acids, may solidify or crystallize at oxidation conditions and/or as the reaction mixture cools. Thus, mixtures of oxidation products may be produced which require further processing to increase the purity of the desired product. For example, in the production of terephthalic acid, the oxidation product is often referred to as crude terephthalic acid because it contains impurities including color bodies and intermediate oxidation products, especially 4-carboxybenzaldehyde (4-CBA). To obtain polymer grade or purified terephthalic acid, various purification steps are known in the art including: washing the crude terephthalic acid with water and/or a solvent, additional oxidation or crystallization steps, and reacting a solution of dissolved crude terephthalic acid with hydrogen at hydrogenation conditions usually including a catalyst comprising palladium and carbon. Often several purification steps are used.

U.S. Pat. No. 2,833,816 discloses processes for oxidizing aromatic compounds to the corresponding aromatic carboxylic acids. A process for the liquid phase oxidation of alkyl aromatic compounds uses molecular oxygen, a metal or metal ions, and bromine or bromide ions in the presence of an acid. The metals may include cobalt and/or manganese. Exemplary acids are lower aliphatic mono carboxylic acids containing 1 to 8 carbon atoms, especially acetic acid.

U.S. Pat. No. 6,355,835 discloses a process for the preparation of benzene dicarboxylic acids by liquid phase oxidation of xylene isomers using oxygen or air by oxidizing in the presence of acetic acid as a solvent, a cobalt salt as a catalyst, and an initiator. The oxidation step is followed by flashing the reaction mixture to remove volatile substances and cooling and filtering the material to get crude benzene di-carboxylic acid as a solid product and a filtrate. Recrystallizing the crude benzene di-carboxylic acid to obtain at least 99% purity and recycling of the filtrate are also disclosed.

U.S. Pat. No. 7,094,925 discloses a process for preparing an alkyl-aromatic compound. The process includes mixing an oxidizing agent or sulfur compound in the presence of an ionic liquid. Air, dioxygen, peroxide, superoxide, or any other form of active oxygen, nitrite, nitrate, and nitric acid or other oxides or oxyhalides of nitrogen (hydrate or anhydrous) can be used as the oxidizing agent. The process is typically carried out under Bronstead acidic conditions. The oxidation is preferably performed in an ionic liquid containing an acid promoter, such as methanesulfonic acid. The product is preferably a carboxylic acid or ketone or intermediate compound in the oxidation, such as an aldehyde, or alcohol.

U.S. Pat. No. 7,985,875 describes a process for preparing an aromatic polycarboxylic acid by liquid phase oxidation of a di- or tri-substituted benzene or naphthalene compound. The process involves contacting the aromatic compound with an oxidant in the presence of a carboxylic acid solvent, a metal catalyst, and a promoter in a reaction zone. The promoter is an ionic liquid comprising an organic cation and a bromide or iodide anion. The promoter is used in a concentration range of about 10 to about 50,000 ppm (based on solvent) with a preferred range of 10-1,000 ppm. No other promoters, such as bromine-containing compounds, need to be used in the process. The process produces crude terephthalic acid (CTA) having 1.4-2.2% 4-CBA. Purification of the CTA is required to obtain purified terephthalic acid (PTA).

US 2010/0174111 describes a process for purifying aryl carboxylic acids, such as terephthalic acid. The impure acid is dissolved or dispersed in an ionic liquid. A non-solvent (defined as a molecular solvent for which the ionic solvent has high solubility and for which the aryl carboxylic acid has little or no solubility) is added to the solution to precipitate the purified acid.

U.S. Pat. No. 7,692,036, 2007/0155985, 2007/0208193, and 2010/0200804 disclose a process and apparatus for carrying out the liquid-phase oxidation of an oxidizable compound. The liquid phase oxidation is carried out in a bubble column reactor that provides for a highly efficient reaction at relatively low temperatures. When the oxidized compound is para-xylene, the product from the oxidation reaction is CTA which must be purified. Purification is said to be easier than for conventional high temperature processes.

Recently, a method was disclosed to utilize a solvent comprising ionic liquid which significantly reduces the amount of 4-CBA in products. For example, US 2012/0004449, 2012/0004450, 2012/0004454, each of which is incorporated herein by reference, describe processes and mixtures for oxidizing alkyl aromatic compounds. The process involves forming a mixture comprising the alkyl-aromatic compound, a solvent, a bromine source, and a catalyst, and contacting the mixture with an oxidizing agent at oxidizing conditions to produce an oxidation product comprising at least one of an aromatic aldehyde, and aromatic alcohol, an aromatic ketone, and an aromatic carboxylic acid. The solvent comprises a carboxylic acid having one to seven carbon atoms and an ionic liquid selected from an imidazolium ionic liquid, a pyridinium ionic liquid, a phosphonium ionic liquid, a tetra alkyl ammonium ionic liquid, or combinations thereof.

US 2012/0004456, which is incorporated herein by reference, describes a process for purifying crude terephthalic acid with a solvent comprising an ionic liquid at purifying conditions to produce a solid terephthalic acid product having a concentration of contaminant lower than the first concentration.

However, under some thermal and oxidation conditions, the composition of the ionic liquid may change. When ionic liquids are used in applications which include oxidation of methyl aromatics and purification of terephthalic acid, changes in the ionic liquid composition could lead to changes in reactivity of the substrate being oxidized. This could cause significant problems in using recycled ionic liquid in an alkyl aromatic oxidation process.

There is a need in the art for improved ionic liquid compositions that can be used in oxidation processes and for processes for making the ionic liquid compositions.

### SUMMARY OF THE INVENTION

One aspect of the invention is a thermally treated A, B-imidazolium ionic liquid solvent composition. In one embodiment, the thermally treated A, B-imidazolium ionic liquid solvent composition includes A, B-imidazolium cation and an anion; A-imidazolium cation and an anion; B-imidazolium cation and an anion; with the proviso that the thermally treated A, B-imidazolium ionic liquid solvent composition does not contain alkyl aromatic compounds or oxidation products of alkyl aromatic compounds; wherein the thermally treated ionic liquid solvent composition has been thermally treated at a temperature of at least about 160° C. for at least about 15 min in the absence of an alkyl aromatic compound, and wherein A and B are independently selected from alkyl groups, substituted alkyl groups, aryl groups, or substituted aryl groups having from 1 to 12 carbons.

Another aspect of the invention is a process for forming a thermally treated A, B-imidazolium ionic liquid solvent composition. In one embodiment, the process includes thermally treating an A, B-imidazolium ionic liquid at a temperature of at least about 160° C. for at least about 15 min in the absence of an alkyl aromatic compound to form the thermally treated A, B-imidazolium ionic liquid solvent composition which includes A, B-imidazolium cation and an anion; A-imidazolium cation and an anion; B-imidazolium cation and an anion; and wherein A and B are independently selected from alkyl groups, substituted alkyl groups, aryl groups, or substituted aryl groups having from 1 to 12 carbons; with the proviso that the thermally treated A, B-imidazolium ionic liquid solvent composition does not contain alkyl aromatic compounds or oxidation products of alkyl aromatic compounds.

### BRIEF DESCRIPTION OF THE DRAWINGS

FIG. 1 is an illustration of reaction products formed from heating or heating and oxidizing 1-butyl-3-methylimidzolium.

FIG. 2 is a graph showing the thermogravometric analysis (TGA) analysis of N,N-dibutylimidazolium bromide, 1-butyl-3-methylimidzolium bromide, and 1-butyl-3-methylimidzolium acetate.

### DETAILED DESCRIPTION OF THE INVENTION

Ionic liquid compositions derived from imidazolium based ionic liquids that have been thermally treated or thermally and oxidatively treated are disclosed. These compositions can be utilized as the medium for oxidation of alkylaromatic compounds and their oxidized derivatives in processes, such as the ones disclosed in US 2012/0004450 and US 2012/0004449, or used as a medium for purification of aromatic carboxylic acids, such as the process disclosed in US 2012/0004456, for example.

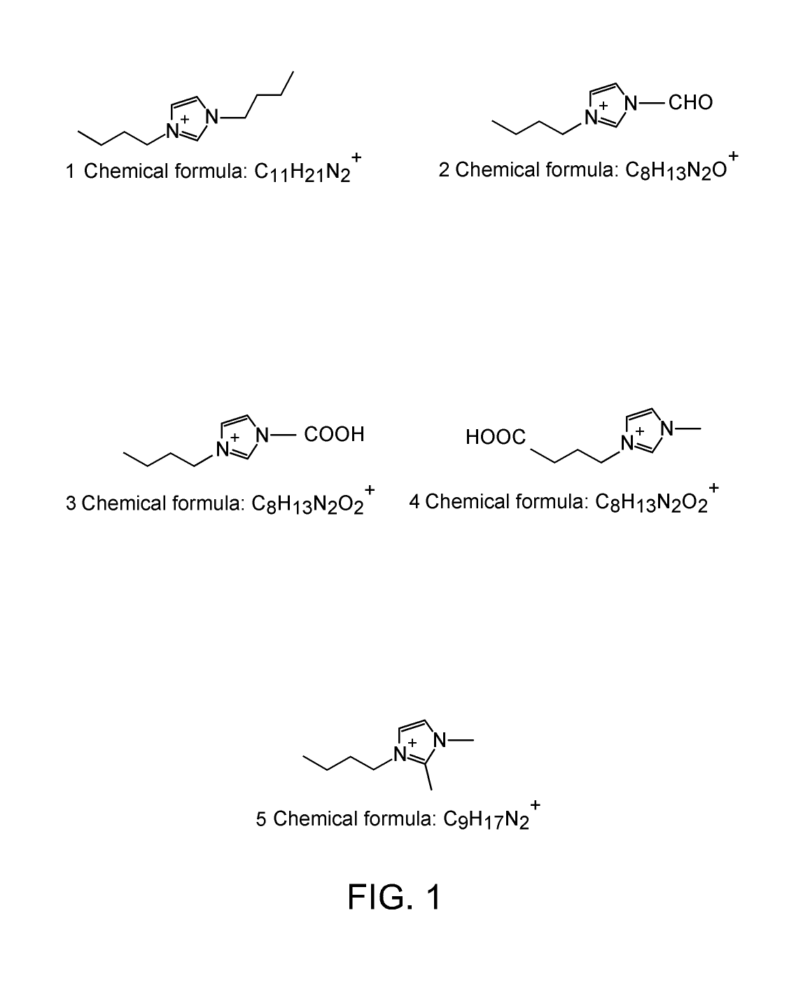

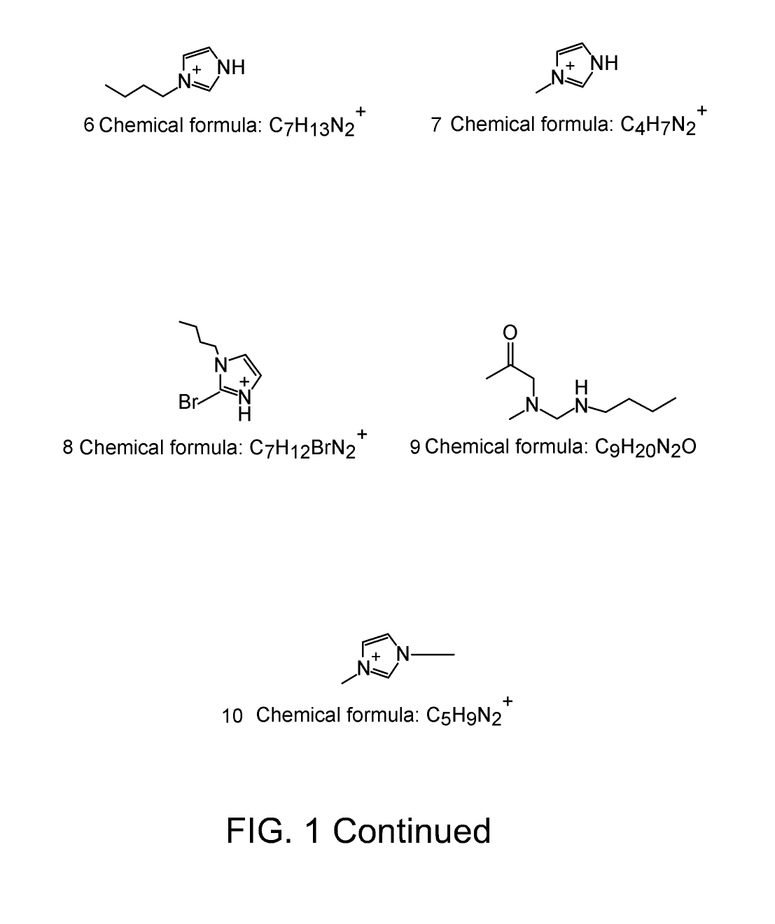

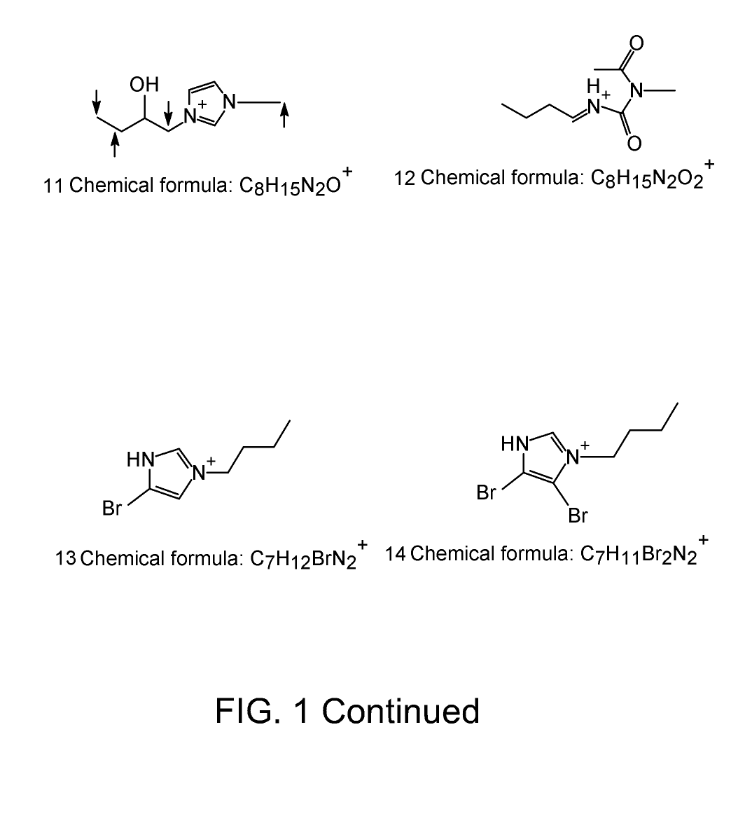

It was discovered that imidazolium based ionic liquids react to form a variety of compounds due to heating, whether in the presence or absence of oxidizing agents. For example, when 1-butyl-3-methylimidazolium is thermally treated and optionally oxidized, in addition to the original components of the mixture, the mixture can contain one or more of 1,3-dibutylimidazoium, N-Butylimidazolium, N-Methylimidazolium, isomers of bromine substituted butyl-imidazolium where one or two bromines are substituents of carbon atoms in the imidazolium ring, and isomers of butyl-dimethylimidazolium, as shown in FIG. 1.

The imidazolium based ionic liquids can be thermally treated at a temperature of at least about 160° C. for at least about 15 min. Higher temperatures form the reaction products in less time than is required at lower temperatures. The temperature can be at least about 160° C., or at least about 170° C., or at least about 180° C., or at least about 190° C., or at least about 200° C., or at least about 205° C., or at least about 210° C., or at least about 215° C., or at least about 220° C., or at least about 225° C., or at least about 250° C., or at least about 275° C., or at least about 300° C. The reaction time can be at least about 15 min, or at least about 30 min, or at least about 45 min, or at least about 1 hr, or at least about 2 hr, or at least about 3 hr, or at least about 5 hr, or at least about 7 hr, or at least about 10 hr, or at least about 12 hr, or at least about 15 hr, or at least about 17 hr, or at least about 20 hr, or at least about 25 hr, or at least about 30 hr.

The thermal treatment is performed in the absence of alkyl aromatic compounds, and the thermally treated imidazolium based ionic liquid solvent composition does not include alkyl aromatic compounds or oxidation products of alkyl aromatic compounds.

A thermally treated A, B-imidazolium ionic liquid solvent composition can include A, B-imidazolium cation and an anion, A-imidazolium cation and an anion, and B-imidazolium cation and an anion. A and B are independently selected from alkyl groups, substituted alkyl groups, aryl groups, or substituted aryl groups having from 1 to 12 carbons. A and B can be the same alkyl group, or they can be different alkyl groups. In some embodiments, A and B both contain at least 2 carbons. The alkyl or aryl groups can be substituted with additional functional groups such as halides, other alkyl or aryl groups, carboxylate groups, esters, ketones, hydroxyl groups, amines, nitrate groups, ethers, sulfate groups, alkyl sulfate groups, phosphate groups, or alkylphosphate groups.

In some embodiments, the composition can include A, A-imidazolium cation and an anion, B, B-imidazolium cation and an anion, or both.

The anions for the various imidazolium cations can be halides, carboxylates, or acetates, for example.

The composition can be oxidized in addition to being thermally treated. The oxidation can take place at the same time as the thermal treatment, or it could take place after the thermal treatment. The oxidation occurs at elevated temperature. i.e., above about 160° C., or above about 170° C., or above about 180° C., or above about 190° C. Suitable oxidizing agents for the process provide a source of oxygen atoms to oxidize the ionic liquid at the oxidation conditions employed. Examples of oxidizing agents include peroxides, superoxides, and nitrogen compounds containing oxygen such as nitric acids. In an embodiment, the oxidizing agent is a gas comprising oxygen, e.g. air, molecular oxygen, and nitrogen dioxide. The gas may be a mixture of gases. The amount of oxygen used in the process is preferably in excess of the stoichiometric amount required for the desired oxidation process. In an embodiment, the amount of oxygen contacted with the mixture (measured as mol of O₂ per mol of carbon in the ionic liquid) ranges from about 0.3 to about 100. Optionally, the amount of oxygen contacted with the mixture may range from about 0.5 to about 30, or from about 0.5 to about 10, or from about 0.5 to about 5, or from about 2 to about 30.

Typically, the oxidation conditions also include a metal catalyst and/or a bromide source. The metal catalyst for oxidation can include at least one of cobalt, manganese, titanium, chromium, copper, nickel, vanadium, iron, molybdenum, tin, cerium and zirconium.

FIG. 1 shows various possible reaction products of 1-butyl-3-methylimidzolium bromide. Compounds (1), (6), (7), and (10) form in both non-oxidizing conditions and oxidizing conditions. Compounds (5), (8), (13), and (14) only formed under oxidizing conditions. Small amounts of compounds (2), (3), (9), (11), and (12) formed in some oxidizing conditions, but not all.

Some reaction products form relatively quickly, while others form more slowly. The amount of some products, such as compound (6), increases gradually. Others, such as compound (7), increase gradually for a period of time, and then increase significantly after that period, such as overnight.

In some embodiments, the ionic liquid cation portion of the composition includes less than about 96 mol % 1-butyl-3-methyl imidazolium cation, after thermal treatment at a temperature of at least about 160° C. for at least about 15 min, or less than about 95 mol %, or less than about 93 mol %, or less than about 90 mol %, or less than about 85 mol %, or less than about 80 mol %, or less than about 75 mol %, or less than about 70 mol %, or less than about 65 mol %, or less than about 60 mol %, or less than about 55 mol %, or less than about 50 mol %, or less than about 45 mol %.

In some embodiments, the ionic liquid cation portion of the composition includes at least about 3 mol % methyl imidazolium cation after thermally treatment at a temperature of at least about 160° C. for at least about 15 min, or at least about 4 mol %, or at least about 5 mol %, or at least about 7 mol %, or at least about 10 mol %, or at least about 15 mol %, or at least about 20 mol %.

In some embodiments, the ionic liquid cation portion of the composition includes at least about 0.2 mol % butyl imidazolium cation after thermally treatment at a temperature of at least about 160° C. for at least about 15 min, or at least about 0.5 mol %, or at least about 1 mol %, or at least about 2 mol %, or at least about 3 mol %, or at least about 5 mol %, or at least about 10 mol %, or at least about 15 mol %, or at least about 20 mol %, or at least about 25 mol %, or at least about 30 mol %.

In some embodiments, the ionic liquid cation portion of the composition includes at least about 0.3 mol % dimethyl imidazolium cation after thermally treatment at a temperature of at least about 160° C. for at least about 15 min, or at least about 0.5 mol %, or at least about 1 mol %, or at least about 1.5 mol %, or at least about 2 mol %, or at least about 3 mol %, or at least about 5 mol %.

In some embodiments, the ionic liquid cation portion of the composition includes at least about 0.2 mol % dibutyl imidazolium cation after thermally treatment at a temperature of at least about 160° C. for at least about 15 min, or at least about 0.3 mol %, or at least about 0.5 mol %, or at least about 0.7 mol %, or at least about 1 mol %, or at least about 1.5 mol %.

In some embodiments, the ionic liquid cation portion of the composition includes at least about 0.5 mol % 1-butyl-2,3-dimethyl imidazolium cation after thermally treatment at a temperature of at least about 160° C. for at least about 15 min, or at least about 1 mol %, or at least about 1.5 mol %, or at least about 2 mol %, or at least about 3 mol %, or at least about 4 mol %.

In some embodiments, the ionic liquid cation portion of the composition includes less than about 96 mol % 1-butyl-3-methyl imidazolium cation and an anion; at least about 3 mol % methyl imidazolium cation and an anion; and at least about 0.2 mol % butyl imidazolium cation and an anion.

In some embodiments, the ionic liquid cation portion of the composition includes less than about 95 mol % 1-butyl-3-methyl imidazolium cation; at least about 4.5 mol % methyl imidazolium cation; and at least about 0.9 mol % butyl imidazolium cation after treatment for at least about 2 hrs.

In some embodiments, the ionic liquid cation portion of the composition includes less than about 80 mol % 1-butyl-3-methyl imidazolium cation; at least about 5 mol % methyl imidazolium cation; and at least about 10 mol % butyl imidazolium cation after treatment for at least about 23 hrs.

In some embodiments, the ionic liquid cation portion of the composition includes less than about 50 mol % 1-butyl-3-methyl imidazolium cation; at least about 15 mol % methyl imidazolium cation; at least about 20 mol % butyl imidazolium cation; at least about 3 mol % dimethyl imidazolium cation; and at least about 0.5 mol % dibutyl imidazolium cation after treatment for at least about 68 hrs.

When the ionic liquid is oxidized in the presence of a oxidizing catalyst and in the absence of an alkyl aromatic compound, the ionic liquid cation portion of the composition can include less than about 90 mol % 1-butyl-3-methyl imidazolium cation; at least about 5 mol % methyl imidazolium cation; at least about 0.6 mol % butyl imidazolium cation; at least about 0.4 mol % dimethyl imidazolium cation; and at least 0.6 mol % 1-butyl-2,3-dimethyl imidazolium cation after treatment for at least about 3 hrs. In other embodiments, the ionic liquid cation portion of the composition can include less than about 75 mol % 1-butyl-3-methyl imidazolium cation; at least about 15 mol % methyl imidazolium cation; at least about 1.5 mol % butyl imidazolium cation; at least about 1.0 mol % dimethyl imidazolium cation; and at least 1.5 mol % 1-butyl-2,3-dimethyl imidazolium cation after treatment for at least about 22 hrs.

A composition containing a dialkylimidazolium, such as N,N-dibutylimidazolium, where the alkyl groups contain greater than one carbon, has the possible advantage of being more stable than 1-butyl-3-methylimidazolium at high temperature, and less likely to form the product N-methylimidazolium or other imidazoliums with exposed nitrogen which are more capable of chelating metals and possibly inhibiting the reaction. These mono-N-alkylimidazoliums also easily convert to the uncharged state by deprotonation and become more susceptible to evaporation and possibly oxidation.

In addition to the imidazolium components, the composition can include other components including, but not limited to carboxylic acid, dissolved ionic solids, water, and catalyst.

In some embodiments, the composition can also comprise a carboxylic acid. The carboxylic acid is generally present in an amount of about 30 wt % to about 99.5 wt % on the basis of the entire composition. The carboxylic acid desirably has from 1 to 7 carbon atoms. In an embodiment, the carboxylic acid comprises acetic acid. The solvent may contain more than one carboxylic acid. For example, the solvent may further comprise benzoic acid. In another embodiment, the carboxylic acid of the solvent is acetic acid.

In some embodiments, an ionic solid, such as ammonium acetate (NH₄OAc) and/or ammonium bromide (NH₄Br), can be dissolved in the mixture. Alternatively, a material which is capable of forming an ionic salt in solution can be added. The material can form the ionic salt in solution by combining with ions present in the solution. For example, in a solution containing bromide (for example in the form of HBr) or acetate ions (for example, in the form of acetic acid), ammonia could combine with the bromide or acetate ions forming ammonium bromide or ammonium acetate. The ionic solid (or material capable of forming the ionic salt) is generally present in an amount of 0 to about 30 wt %, or 0 to about 20 wt %.

Optionally, the composition may further comprise water. The water may be added to the mixture or generated in the mixture during the reaction. Water is generally present in an amount of 0 to 30 wt %, or 0 to about 20 wt %, or 0 to about 10 wt %, or 0 to about 5 wt %.

A catalyst can be present during the thermal treatment and/or the oxidation. The catalyst can be of the same type used for the oxidation of alkylaromatic compounds to aromatic carboxylic acids. The catalyst can comprise at least one of cobalt, manganese, titanium, chromium, copper, nickel, vanadium, iron, molybdenum, tin, cerium and zirconium. In an embodiment, the catalyst comprises cobalt and manganese. The metal may be in the form of an inorganic or organic salt. For example, the metal catalyst may be in the form of a carboxylic acid salt, such as, a metal acetate and hydrates thereof. Exemplary catalysts include cobalt (II) acetate tetrahydrate and manganese (II) acetate, individually or in combination. In an embodiment, the amount of manganese (II) acetate is less than the amount of cobalt (II) acetate tetrahydrate by weight. The catalyst is generally present in an amount of about 0.1% to about 2%, or about 0.1% to about 1% or about 0.1% to about 0.5%.

Bromine sources are generally recognized in the art as being catalyst promoters and can be included in some embodiments. Suitable bromine sources include, but are not limited to, bromine, ionic bromine, e.g. HBr, NaBr, KBr, NH₄Br; and/or organic bromides which are known to provide bromide ions at the oxidation conditions, such as, benzylbromide, mono and di-bromoacetic acid, bromoacetyl bromide, tetrabromoethane, ethylene di-bromide. In an embodiment, the bromine source comprises or consists essentially of or consists of hydrogen bromide. The bromine source is generally present in an amount of about 0.2 to about 1.5%. The amount of bromine source does not include the amount of bromine in the ionic liquid.

The thermal treatment and/or oxidizing steps may be practiced in laboratory scale experiments through full scale commercial operations. The process may be operated in batch, continuous, or semi-continuous mode. The thermal treatment and/or oxidizing steps can take place in various ways. The order of addition of the components (e.g., ionic liquid, carboxylic acid, bromine source, catalyst, and oxidizing agent) is not critical. For example, the components can be added individually, or two or more components may be combined or mixed before being combined or mixed with other components.

The thermally treated imidazolium based ionic liquid solvent composition can be used in processes for the oxidation of alkyl aromatic compounds. Such processes are described in US 2012/0004450 and US 2012/0004449, for example. The thermally treated imidazolium based ionic liquid solvent composition is used as the solvent for the reaction, and it can be recycled and reused. The composition is stable over time, most likely because the treatment results in a composition that is close to the equilibrium composition at reaction temperature. If proper reaction time and temperature are chosen, compositions can be obtained which have more stable components such as dibutylimidazolium (compound 1) instead of BMIm.

The oxidation of the alkyl aromatic compound involves contacting an alkyl aromatic compound, the thermally treated imidazolium based ionic liquid solvent composition, a bromine source, a catalyst, and an oxidizing agent to produce at least one of an aromatic alcohol, an aromatic aldehyde, an aromatic ketone, and an aromatic carboxylic acid.

Suitable alkyl aromatic compounds or feeds to be oxidized include aromatic compounds comprising at least one benzene ring having at least one alkyl group. Methyl, ethyl, and isopropyl alkyl groups are preferred alkyl groups, although other alkyl groups can be used if desired. In an embodiment, the alkyl aromatic compound is selected from toluene, para-xylene, ortho-xylene, and meta-xylene. The feed may comprise more than one alkyl aromatic compound. As the oxidation reaction generally proceeds through successive degrees of oxidization, suitable feed compounds also include partially oxidized intermediates relative to the desired oxidized product. For example, in the production of terephthalic acid, the alkyl aromatic feed may comprise para-toluic acid and/or 4-carboxybenzaldehyde (4-CBA).

The catalyst, bromine source, and oxidizing agent can be the same ones described above.

Conventional, liquid phase oxidation reactors as known in the art may be used to practice the invention. Examples include vessels, which may have one or more mechanical agitators, and various bubble column reactors such as those described in U.S. Pat. No. 7,692,036. It is also known to design, operate, and control such reactors and the oxidation reaction for the oxidation conditions employed including, e.g., the temperature, pressure, liquid and gas volumes, and corrosive nature of the liquid and gas phases where applicable. See, e.g. U.S. Pat. No. 7,692,036 and U.S. Pat. No. 6,137,001.

The contacting step[s] can take place under oxidizing conditions, if desired. Suitable oxidizing conditions generally include a temperature ranging from about 125° C. to about 275° C. and a pressure ranging from about atmospheric, i.e. 0 MPa(g), to about 6 MPa(g) and a residence time ranging from about 5 seconds to about 2 weeks. That is, the mixture has a temperature and a pressure within these ranges and may be maintained within these ranges for a period of time within the residence time range. In another embodiment, the temperature ranges from about 175° C. to about 225° C.; and the temperature may range from about 190° C. to about 235° C. In an embodiment, the pressure ranges from about 1.2 MPa(g) to about 6.0 MPa(g); and the pressure may range from about 1.5 MPa(g) to about 6.0 MPa(g). In a further embodiment, the residence time ranges from about 10 minutes to about 12 hours. The oxidation temperature, pressure and residence time may vary based on a variety of factors including for example, the reactor configuration, size, and whether the process is, batch, continuous, or semi-continuous. An oxidation condition may also vary based on other oxidation conditions. For example, use of a particular temperature range may enable use of a different residence time range.

The product can be crystallized and separated using known processes, such as those described in US 2012/0004450 and US 2012/0004449, for example.

### EXAMPLES

In addition to the imidazolium derivatives, the compositions typically also contain acetic acid, ammonium acetate, water, 1-butyl-3-methylimidazolium bromide (BMImBr), 1-butyl-3-methylimidazolium acetate (BMImOAc), and a catalyst consisting of cobalt and manganese acetates and hydrobromic acid. Unless otherwise noted below, these mixtures are prepared from a mixture containing approximately 50 wt % acetic acid, 10% BMImOAc, 20% BMImBr, 20% ammonium acetate, and less than 1% water.

### Thermal Reaction to Form “Aged” Ionic Liquid

A composition containing ionic liquid components from among BMIm, compounds (6), (1), (7) and (10) can be obtained by heating a mixture containing BMIm under an inert gas. This mixture can then be used as a solvent for para-xylene oxidation to obtain a solid terephthalic acid containing product with less than 500 ppm 4-CBA.

### Example 1

A starting mixture of 75.7 g acetic acid, ionic liquid (containing both 29.87 g BMIm bromide and 14.98 g BMIm acetate), 15 g ammonium acetate, and 0.6 g water was heated in a pressurized titanium autoclave under flowing nitrogen at 400 psig. (Other non-reactive gases could be used, if desired, and the gas does not need to be flowing) The mixture was first heated to 180° C. for two hours, and a sample was collected from the vessel. This sample contained no significant ionic liquid components (MS signal>10e4 counts) other than BMIm. The mixture was heated for an additional 68 hours and analyzed after cooling. This process produces BMIm reaction products that are accessible by thermal reaction. High performance liquid chromatography (HPLC) with UV-vis detection showed that no overall loss of imidazolium compounds occurred. The mixture had 43% BMIm, 32% compound (6), 2% compound (1), 18% compound (7) and 5% compound (10) as a percentage of moles of ionic liquid cations. The quantification of compound (10) is an estimate assuming a similar UV-vis molar absorptivity as other cations.

Subsequently, the product of this reaction was used as a solvent for para-xylene oxidation. The solvent included 80 g of the product of the above reaction, and 20 g of a mixture of acetic acid, BMImBr, BMImOAc and ammonium acetate (in the same proportion as original starting material). A catalyst including 0.4 g HBr, 0.8 g Cobalt(II) acetate tetrahydrate and 0.8 g Manganese(II) acetate was also added, along with 20 g para-xylene. The reaction was run by heating to a target temperature of 215° C. with 2500 sccm air flow for three hours, although the temperature dropped to 210° C. after the initial exotherm which lasted approximately 1 hour. After isolating the products by filtration and washing, the solid portion of the products of this reaction (as analyzed by HPLC) contained 463 ppm 4-CBA, 4641 ppm toluic acid and 823 ppm toluamide, with the remainder being terephthalic acid and terephthalic acid-monoamide in a ratio of 2.4:1.

### Example 2

A starting mixture of 124.5 g acetic acid, ionic liquid (containing both 49.8 g BMIm bromide and 24.9 g BMIm acetate), 49.8 g ammonium acetate, and 1.0 g water was heated in a pressurized titanium autoclave under flowing nitrogen at 400 psig. The mixture was first heated to 200° C. for two hours, and samples were collected from the vessel at 0, 15, 60 and 120 minutes after reaching temperature. After the period at 200° C., the reactor was heated to 215° C. for 19 hours, followed by 3 hours at 200° C. The composition of each sample was quantified, and the results are shown in Table 2. The numbers are in moles of ionic liquid cation normalized to total moles of ionic liquid cations. A portion of the BMIm ionic liquid cations is first converted to compound (7). Most of this conversion occurred at a temperature below 200° C., during the reactor's heating period, and then occurred much more slowly as the reaction progressed to a total of 7.4% after the complete temperature program. Formation of compound (6) occurred only after 200° C. temperature was reached. It formed at relatively constant rate thereafter. Compound (10) was observed only after 1 hour at the 215° C. condition. Note that quantification of compound (10) is an estimate that assumes similar molar absorptivity to other ionic liquid cations. Finally, compound (1) formed after reaction overnight at 215° C.

TABLE 1
composition of ionic liquid portion of thermally aged
ionic liquid as function of time and temperature

| | Time | | | | | | |
|---|---|---|---|---|---|---|---|
| | at | Total | | | | | |
| | temp | time* | | | | | |
| Temperature | (min) | (min) | BMIM | (7) | (6) | (1) | (10)** |
| 200 | 0 | 0 | 96.5% | 3.2% | 0.2% | 0.0% | 0.0% |
| 200 | 15 | 15 | 95.5% | 4.3% | 0.3% | 0.0% | 0.0% |
| 200 | 60 | 61 | 94.9% | 4.5% | 0.5% | 0.0% | 0.0% |
| 200 | 120 | 120 | 94.6% | 4.5% | 0.9% | 0.0% | 0.0% |
| 215 | 0 | 140 | 94.3% | 4.6% | 1.1% | 0.0% | 0.0% |
| 215 | 15 | 155 | 93.9% | 4.7% | 1.5% | 0.0% | 0.0% |
| 215 | 60 | 215 | 92.7% | 4.8% | 2.2% | 0.0% | 0.3% |
| 215 | 121 | 336 | 90.6% | 5.0% | 3.6% | 0.0% | 0.8% |
| 215 | 1019 | 1355 | 74.3% | 7.0% | 17.4% | 0.5% | 0.8% |
| 200 | 185 | 1557 | 71.1% | 7.3% | 19.9% | 0.5% | 1.1% |

*where time 0 is when reactor reached 200° C.
**estimated assuming similar molar absorptivity as other ionic liquid cations

Subsequently, the product of this reaction was used as a solvent for para-xylene oxidation. The solvent was 100 g of the product of the above reaction. A catalyst including 0.4 g HBr, 0.8 g Cobalt(II) acetate tetrahydrate and 0.8 g Manganese(II) acetate was also added, along with 20 g para-xylene. The reaction was run by heating to a target temperature of 215° C. with 2500 sccm air flow for three hours, although the temperature dropped to 210° C. after the initial exotherm which lasted approximately 1 hour. After isolating the products by filtration and washing, the solid portion of the products of this reaction (as analyzed by HPLC) contained 292 ppm 4-CBA, 2544 ppm toluic acid and 689 ppm toluamide, with the remainder being terephthalic acid and terephthalic acid-monoamide in a ratio of 2.2:1.

### Comparative Example 1

A pX oxidation reaction was run under similar conditions to examples 1 and 2 but using fresh ionic liquid instead of first thermally aging the ionic liquid and then using it for pX oxidation. 20 g para-xylene was added to a mixture of 50 g acetic acid, 20 g BMImBr, 20 g ammonium acetate, 10 g BMImOAc, 0.4 g HBr, 0.4 g water, 0.8 g cobalt(II) acetate tetrahydrate, and 0.6 g manganese(II) acetate. The reaction occurred under flowing air at 2500 sccm, 400 psig and 215° C., although the temperature dropped to 210° C. after the initial exotherm which lasted approximately 1 hour. After isolating the products by filtration and washing, the solid portion of the products of this reaction (as analyzed by HPLC) contained 445 ppm 4-CBA, 502 ppm benzoic acid, 9022 ppm toluic acid, and 1592 ppm toluamide, with the remainder being terephthalic acid and terephthalic acid-monoamide in a ratio of 1.7:1.

### Oxidation Reaction to Form “Oxidized-Aged” Ionic Liquid

Oxidation of ionic liquid under para-xylene reaction conditions can be utilized to generate some additional components of this composition, including compound (5). Compounds (2), (11), (8), (13), and (14) were also observed in trace amounts with mass spectrometry detection, but they appeared to be in quantities of less than approximately 1000 ppm based on extracted ion chromatograms from liquid chromatography-mass spectrometry.

### Example 3

In separate examples, ionic liquid was subjected to an oxidative aging treatment for 21 hours at 215° C., similar to the thermal treatment but under flowing air and including Co, Mn, HBr catalyst but no methyl aromatic reactant (or intermediate). This process resulted in generation of all of the products that result from thermal treatment (in examples 1 and 2), plus significant amounts of compound (5), and trace amounts of compounds (2), (8), (11), and (13) observed in mass spectrometry. Compound (1) was also only observed in trace amounts. However, when conducted in the presence of para-xylene, even the trace amounts of oxygenated BMIm, compounds (2) and (11), were not observed. Similar results were obtained when the oxidation was conducted for only 3 hours. High performance liquid chromatography (HPLC) with UV-vis detection shows that no overall loss of imidazolium compounds occurred. The amounts of BMIm, and compounds (6), (1), (5), (7) and (10) as a percentage of moles of ionic liquid cations are shown in Table 2. The quantification of compound (10) is an estimate assuming similar UV-vis molar absorptivity as other cations.

TABLE 2

| Air | Time | Reaction | | | | | | |
|---|---|---|---|---|---|---|---|---|
| and | on | temper- | | | | | | |
| cata- | stream | ature | Cation as mol % of total ionic liquid cations | | | | | |
| lyst? | (hr) | (° C.) | BMIm | (1) | (5) | (6) | (7) | (10) |
| No | 68 | 215 | 43% | 1.9% | 0.0% | 32% | 18% | 4.7% |
| Yes | 3 | 215 | 70% | 0.0% | 4.1% | 5.0% | 19% | 2.0% |
| Yes | 21.5 | 215 | 70% | 0.0% | 2.1% | 2.0% | 25% | 1.6% |

Subsequently, 70 g of the product of this reaction was used as a solvent for para-xylene oxidation, after addition of 0.4 g HBr, 33.8 g acetic acid (to make up for loss during the initial oxidation), and 20 g para-xylene. The reaction was run by heating to a target temperature 215° C. with 2500 sccm air flow for three hours, although the temperature dropped to 206° C. after the initial exotherm. After isolating the products by filtration and washing, the solid portion of the products of this reaction (as analyzed by HPLC) contained 6669 ppm 4-CBA, 19.8% toluic acid and 5.0% toluamide, with the remainder consisting of terephthalic acid and terephthalic acid-monoamide in a ratio of 1.2:1. The high amount of toluic acid and toluamide indicate reaction did not reach full conversion, which may also be the reason for high 4-CBA.

### Synthesized Ionic Liquid Mixture

Instead of generating the components of the ionic liquid composition in this invention, one may add precursors to the reactor which result in formation of some or all of the composition's components.

### Example 4

In one example, a para-xylene oxidation reaction was conducted similar to comparative example 1 but substituting 6.24 g 1-butylimidazole and an additional 3.03 g acetic acid instead of the 10 g BMIm acetate that was used in the comparative example. 1-butylimidazole and acetic acid were intended to form compound (6) and acetate anion upon heating. This resulted in no 4-CBA observed, 1345 ppm toluic acid, 306 ppm toluamide, and a terephthalic acid to terephthalic acid monoamide ratio of 1.7 in the solid products. This example demonstrates that the presence of N-butylimidazolium is not detrimental for obtaining low 4-CBA in the para-xylene oxidation process.

### Example 5

1,3-dibutylimidazolium bromide ionic liquid was synthesized by mixing excess 1-bromobutane with 1-butylimidazole at 150° C. for 5 hours under 230 psig nitrogen (It could also be synthesized in lower yield at room temperature), followed by decreasing the pressure to atmospheric pressure to distill the product. A para-xylene oxidation reaction was conducted similar to comparative example 1 but using 1,3-dibutylimidazolium bromide (1) instead of BMImBr and BMImOAc (where the amount of 1,3 dibutylimidazolium bromide was equal in moles to the total amount of BMImBr and BMImOAc used in comparative example 1). In this reaction, 90 mol % of the recovered ionic liquid was compound (1), demonstrating that this ionic liquid is more stable than BMIm. The remainder was 2% BMIm, 5% compound (6) and 3% compound (5). This resulted in 4388 ppm 4-CBA, 6862 ppm toluic acid, 784 ppm toluamide, and a terephthalic acid to terephthalic acid monoamide ratio of 2.7:1 in the solid products.

In a comparison, using all BMImBr in equivalent molar amount to the amount of compound (1) used above (as opposed to comparative example 1 where some BMImOAc was used), only 74 mol % of the recovered ionic liquid was BMIm. The remainder was 22% compound (7), 1% compound (6) and 3% compound (10). This resulted in 1076 ppm 4-CBA, 199 ppm toluic acid, 352 ppm toluamide and a terephthalic acid to terephthalic acid monoamide ratio of 2.5:1 in the solid products.

### Example 6

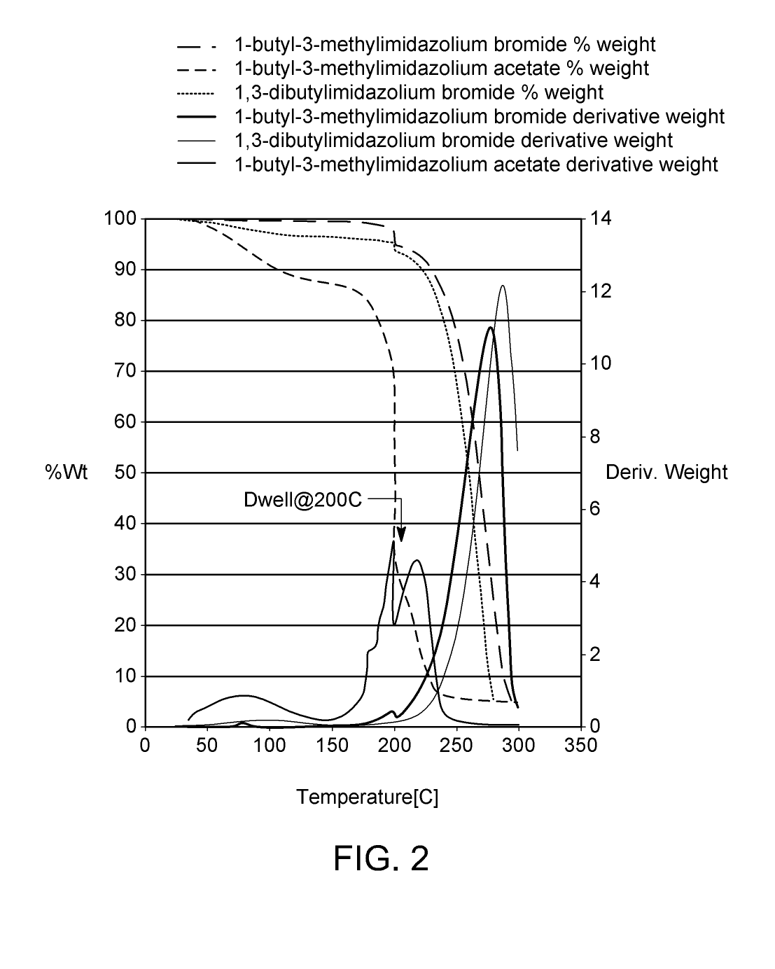

TGA (thermogravometric analysis) experiments demonstrated that N,N-dibutylimidazolium (1) bromide has higher thermal stability than BMImBr or BMImOAc. Ionic liquid was heated in a mixture of 20% O2, 80% helium (by volume) at 5° C./min to 200° C., held at that temperature for 10 min, and then heated at 5° C./min to 300° C. The peak weight loss occurred during the 200° C. hold step for BMImOAc, at 275° C. for BMImBr, and at 286° C. for N,N-dibutylimidazolium (1) bromide, as shown in FIG. 2.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A thermally treated A, B-imidazolium ionic liquid solvent composition comprising:
A, B-imidazolium cation and an anion;
A-imidazolium cation and an anion;
B-imidazolium cation and an anion;
with the proviso that the thermally treated A, B-imidazolium ionic liquid solvent composition does not contain alkyl aromatic compounds or oxidation products of alkyl aromatic compounds; and
wherein the thermally treated ionic liquid solvent composition has been thermally treated at a temperature of at least about 160° C. for at least about 15 min in the absence of an alkyl aromatic compound, and wherein A and B are independently selected from alkyl groups, substituted alkyl groups, aryl groups, or substituted aryl groups having from 1 to 12 carbons.

2. The composition of claim 1 further comprising:
A, A-imidazolium cation and an anion; or
B, B-imidazolium cation and an anion; or
both.

3. The composition of claim 1 wherein the composition comprises:
less than about 96 mol % 1-butyl-3-methyl imidazolium cation and an anion;
at least about 3 mol % methyl imidazolium cation and an anion; and
at least about 0.2 mol % butyl imidazolium cation and an anion;
wherein mol % is based on a total amount of ionic liquid.

4. The composition of claim 1 wherein the composition comprises:
less than about 80 mol % 1-butyl-3-methyl imidazolium cation;
at least about 5 mol % methyl imidazolium cation; and
at least about 10 mol % butyl imidazolium cation;
wherein mol % is based on a total amount of ionic liquid.

5. The composition of claim 1 wherein the composition comprises:
less than about 90 mol % 1-butyl-3-methyl imidazolium cation;
at least about 5 mol % methyl imidazolium cation;
at least about 0.6 mol % butyl imidazolium cation;
at least about 0.4 mol % dimethyl imidazolium cation; and
at least 0.6 mol % 1-butyl-2,3-dimethyl imidazolium cation;
wherein the thermally treated ionic liquid solvent composition has been oxidized in the presence of an oxidation catalyst and in the absence of an alkyl aromatic compound.

6. The composition of claim 5 wherein the oxidation catalyst comprises at least one of cobalt, manganese, titanium, chromium, copper, nickel, vanadium, iron, molybdenum, tin, cerium and zirconium.

7. The composition of claim 6 wherein the oxidation catalyst further comprises a bromine source.

8. The composition of claim 1 further comprising at least one of a carboxylic acid having from 1 to 7 carbon atoms, water, a dissolved ionic solid, or a second oxidation catalyst.

9. The composition of claim 8 wherein the dissolved ionic solid is present and wherein the dissolved ionic solid comprises ammonium acetate.

10. The composition of claim 1 wherein the anion for one or more of the A, B-imidazolium cation, the A-imidazolium cation, or the B-imidazolium cation is selected from halides, carboxylates, or acetate.

11. A process for forming a thermally treated A, B-imidazolium ionic liquid solvent composition comprising:
thermally treating an A, B-imidazolium ionic liquid at a temperature of at least about 160° C. for at least about 15 min in the absence of an alkyl aromatic compound to form the thermally treated A, B-imidazolium ionic liquid solvent composition comprising:
A, B-imidazolium cation and an anion;
A-imidazolium cation and an anion;
B-imidazolium cation and an anion; and
wherein A and B are independently selected from alkyl groups, substituted alkyl groups, aryl groups, or substituted aryl groups having from 1 to 12 carbons;
with the proviso that the thermally treated A, B-imidazolium ionic liquid solvent composition does not contain alkyl aromatic compounds or oxidation products of alkyl aromatic compounds.

12. The process of claim 11 further comprising oxidizing the A, B-imidazolium ionic liquid in the presence of an oxidation catalyst and in the absence of an alkyl aromatic compound.

13. The process of claim 12 wherein the oxidation catalyst comprises at least one of cobalt, manganese, titanium, chromium, copper, nickel, vanadium, iron, molybdenum, tin, cerium and zirconium.

14. The process of claim 11 wherein the thermally treated imidazolium based ionic liquid solvent composition further comprises at least one of a carboxylic acid having from 1 to 7 carbon atoms, water, a dissolved ionic solid, or a second oxidation catalyst.

15. The process of claim 14 wherein the dissolved ionic solid is present and wherein the dissolved ionic solid comprises ammonium acetate.

16. The process of claim 11 wherein the thermally treated A, B-imidazolium ionic liquid solvent composition further comprises:
A, A-imidazolium cation and an anion; or
B, B-imidazolium cation and an anion; or
both.

17. The process of claim 11 wherein the thermally treated A, B-imidazolium ionic liquid solvent composition comprises:
less than about 96 mol % 1-butyl-3-methyl imidazolium cation and an anion;
at least about 3 mol % methyl imidazolium cation and an anion; and
at least about 0.2 mol % butyl imidazolium cation and an anion;
wherein mol % is based on a total amount of ionic liquid.

18. The process of claim 11 wherein the thermally treated A, B-imidazolium ionic liquid solvent composition comprises:
less than about 80 mol % 1-butyl-3-methyl imidazolium cation;
at least about 5 mol % methyl imidazolium cation; and
at least about 10 mol % butyl imidazolium cation;
wherein mol % is based on a total amount of ionic liquid.

19. The process of claim 12 wherein the thermally treated A, B-imidazolium ionic liquid solvent composition comprises:
less than about 90 mol % 1-butyl-3-methyl imidazolium cation and an anion;
at least about 6 mol % methyl imidazolium cation and an anion;
at least about 0.6 mol % butyl imidazolium cation and an anion;
at least about 0.4 mol % dimethyl imidazolium cation and an anion; and
at least 0.6 mol % 1-butyl-2,3-dimethyl imidazolium cation and an anion;
wherein mol % is based on a total amount of ionic liquid.

20. A process for producing an aromatic carboxylic acid from an alkyl aromatic compound comprising:
thermally treating an imidazolium based ionic liquid at a temperature of at least about 160° C. for at least about 15 min in the absence of an alkyl aromatic compound to form a thermally treated imidazolium based ionic liquid solvent composition;
forming a mixture comprising the alkyl aromatic compound, the thermally treated imidazolium based ionic liquid solvent composition, a bromine source, an oxidation catalyst and optionally ammonium acetate; and
oxidizing the alkyl aromatic compound by contacting the mixture with an oxidizing agent at oxidizing conditions to produce a solid oxidation product, the solid oxidation product comprising the aromatic carboxylic acid;
wherein the oxidation catalyst comprises at least one of cobalt, titanium, manganese, chromium, copper, nickel, vanadium, iron, molybdenum, tin, cerium, and zirconium.
